(19) United States
(12) Patent Application Publication
(10) Pub. No.: US 2026/0110697 A1
(43) Pub. Date: Apr. 23, 2026

(54) METHOD FOR ANALYZING A TUMOR-SPECIFIC MARKER

(71) Applicant: Snipping GmbH, Berlin (DE)

(72) Inventor: Nils Martin Nesheim, Berlin (DE)

(21) Appl. No.: 19/360,416

(22) Filed: Oct. 16, 2025

(30) Foreign Application Priority Data
Oct. 18, 2024 (DE) 102024130370.0

(51) Int. Cl. G01N 33/68; G01N 1/40; G01N 1/44; G01N 33/50

(52) U.S. Cl. CPC G01N 33/689; G01N 1/4077; G01N 1/44; G01N 33/5044; G01N 33/5091; G01N 2333/47; G01N 2500/10; G01N 2800/342; G01N 2800/52; G01N 2800/7028

(57) ABSTRACT

A method for analyzing a tumor-specific marker and to the use of an isolate from ejaculate for diagnosing and/or monitoring prostate cancer, and/or for monitoring the success of prostate cancer therapy. The method includes the steps of performing an analysis on the isolate, wherein the tumor-specific marker selected from a protein, DNA, and RNA is analyzed, wherein the isolate was obtained by fractionating ejaculate into a low-density cell population and a high-density cell population, wherein the high-density cell population including mature sperm cells was separated and the isolate includes the low-density cell population, wherein the isolate includes a cell pellet.

### CROSS REFERENCE TO RELATED PATENT APPLICATIONS

This patent application claims the benefit of and priority to German Patent Application 102024130370.0 filed Oct. 18, 2024, the entire contents of which are incorporated herein by reference for all purposes.

### TECHNICAL FIELD

The present invention relates to a method for analyzing a tumor-specific marker and the use of an isolate from ejaculate or an isolate from expressed urine for diagnosing and/or monitoring prostate cancer and/or for monitoring the success of prostate cancer therapy.

### BACKGROUND

Prostate cancer is a common disease in men and one of the most frequent causes of death.

Therefore, methods for diagnosing and monitoring prostate cancer have already been developed.

The clinical diagnosis of prostate cancer includes PSA levels, digital rectal examinations, and core needle biopsies. The clinical benefit of the PSA value as a surrogate parameter is controversial (risk of overtreatment). Biopsies are invasive procedures that involve increased effort, risks, and stress for the patient and are therefore not suitable for high frequency monitoring of disease progression. Usually, tumor metastasis is determined retrospectively in the pathology assessment after prostatectomy, when neighboring lymph nodes are removed and examined for tumor infiltration. This is relevant for the so-called “active surveillance” strategy, an approach with the aim delay prostatectomy as long as possible to maintain the patient's quality of life. However, there is a lack of diagnostic procedures to reliably determine the right time to move from “active surveillance” to an aggressive treatment regimen. This results in risks of over- and undertreatment (i.e., unnecessary surgery on the one hand, or delayed treatment initiation on the other hand).

Non-invasive procedures have obvious advantages over invasive procedures such as core needle biopsies. Therefore, the publication “Seminal Plasma as a Source of Prostate Cancer Peptide Biomarker Candidates for Detection of Indolent and Advanced Disease” by Neuhaus J., Schiffer E., von Wilcke P., Bauer H W., Leung H., et al. (2013, PLoS ONE 8(6): e67514. doi: 10.1371/journal.pone.0067514) recommends the use of plasma from ejaculate for the diagnosis of prostate cancer. The plasma is obtained by centrifuging the ejaculate to remove the cells, including sperm cells. However, the comparatively low protein concentration and the contribution of other glands to the analyte (e.g., from the epididymis) can lead to a dilution effect of disease-related changes pertaining to prostate cancer.

US 2017/0 254 810 A1 also deals with the analysis of seminal fluid. Specifically, it discloses a method for detecting biomarkers for prostate cancer, whereby the seminal fluid is examined for subtypes of prostate-specific antigen (PSA).

The publication “DNA-based detection of prostate cancer in blood, urine, and ejaculates” by GOESSL C. et al. (Annals New York academy of sciences. Circulating nucleic acids in plasma or serum II, Vol. 945, 2001, No. 1. pp. 51-58. ISSN 0077-8923) describes the analysis of a prostate cancer specific marker gene in whole ejaculate containing sperm cells and sedimented cells from expressed prostatic secretions (EPS) in prostate massage urine.

Currently there are a variety of urine-based prostate cancer tests available in the United States, but their application is limited to certain timepoints in the prostate cancer patient journey (i.e. decision about re-biopsy when biopsy is suspected to be false negative). These methods have their limitations, and there is currently no semen-based test available. In summary, the currently established methods for prostate cancer monitoring still have disadvantages and pose the risk of over- and undertreatment. There is therefore still a need for an improved method for diagnosing and/or monitoring prostate cancer. There is also a need for a method for monitoring the success of prostate cancer therapy.

The present invention aims to provide an improved method for analyzing a tumor-specific marker and for diagnosing and/or monitoring prostate cancer, as well as for monitoring the success of prostate cancer therapy.

### SUMMARY OF THE INVENTION

The efforts to solve this problem have resulted in a first aspect, a method for analyzing a tumor-specific marker. The method comprises: performing an analysis on an isolate, wherein a tumor-specific marker selected from a protein, DNA, and RNA is analyzed, wherein the isolate was obtained by fractionating ejaculate into a low-density cell population and a high-density cell population, wherein the high-density cell population comprising mature sperm cells was separated, and the isolate comprises the low-density cell population.

The efforts to solve the task mentioned at the outset result in a second aspect, a method for analyzing a tumor-specific marker, wherein the method comprises the following steps:
1) Separating ejaculate into a low-density cell population and a high-density cell population, wherein the high-density cell population comprising mature sperm cells is separated and an isolate comprising the low-density cell population is obtained,
2) performing an analysis on the isolate, wherein a tumor-specific marker selected from a protein, DNA, and RNA is analyzed.

The first and second aspects differ in that the method from the second aspect comprises fractionating ejaculate to obtain the isolate, whereas the isolate is the starting point for the method from the first aspect.

The efforts to solve the task mentioned at the outset result in a third aspect, a method for analyzing a tumor-specific marker, wherein the method comprises: performing an analysis on an isolate, wherein a tumor-specific marker selected from a protein, DNA, and RNA is analyzed, wherein the isolate was obtained by fractionating expressed prostatic secretions (EPS) into a cellular fraction and a cell-poor (“paucicellular”) fraction, wherein the cell-poor fraction was separated and the isolate comprises the cellular fraction.

The efforts to solve the task mentioned at the outset result in a fourth aspect, a method for analyzing a tumor-specific marker, wherein the method comprises the following steps:
1) Separating expressed prostatic secretions (EPS) into a cellular fraction and a cell-poor fraction, wherein the cell-poor (“paucicellular”) fraction is separated and an isolate comprising the cellular fraction is obtained,
2) performing an analysis on the isolate, wherein a tumor-specific marker selected from a protein, DNA, and RNA is analyzed.

The third and fourth aspects differ in that the method according to the fourth aspect comprises fractionating expressed prostatic secretions (EPS) to form the isolate, whereas the isolate is the starting point in the method according to the third aspect.

The efforts to solve the task mentioned at the outset result in a fifth aspect, a method for analyzing a tumor-specific marker, wherein the method comprises: performing an analysis on an isolate, wherein a tumor-specific marker selected from a protein, DNA, and RNA is analyzed, wherein the isolate was obtained by fractionating ejaculate from a vasectomized person into an isolate of a cellular fraction and an isolate of a cell-poor fraction, wherein the analysis is performed on the isolate of the cellular fraction and/or the isolate of the cell-poor fraction.

The efforts to solve the task mentioned at the outset result in a sixth aspect, a method for analyzing a tumor-specific marker, wherein the method comprises the following steps:
1) Separating ejaculate from a vasectomized person into a cellular fraction and a cell-poor fraction, whereby an isolate of the cellular fraction and an isolate of the cell-poor fraction are formed from ejaculate from a vasectomized person,
2) Performing an analysis on the isolate of the cellular fraction and/or the isolate of the cell-poor fraction, wherein a tumor-specific marker selected from a protein, DNA, and RNA is analyzed.

The fifth and sixth aspects differ in that the method according to the sixth aspect comprises fractionating ejaculate from a vasectomized person to form the isolate, whereas the isolate is the starting point in the method according to the fifth aspect.

The idea underlying the invention is to achieve a concentration of disease-relevant markers and to eliminate signals without significance (background) by isolating and analyzing the cellular fraction from semen or expressed urine, except for mature sperm cells, which are to be separated from the analyte by the described method. The isolation of cells from semen or expressed prostatic secretions (EPS) aims to provide the liquid biopsies with the analytical properties and advantages of a tissue biopsy, so to speak. The method further aims to enable closer prostate cancer monitoring without biopsies, providing meaningful information about disease progression and treatment success. The method aims to provide a simple and cost-effective test allowing prostate cancer monitoring beyond the currently established clinical care. Physicians, laboratories, and patients are to be provided with a tool that allows them more clarity and informed decision making during the “active surveillance” treatment approach for prostate cancer. Furthermore, the method aims to help identify aggressive disease entities with a high risk for metastasis in a timely manner. This can prevent premature and unnecessary surgical interventions (known as “overtreatment”) but also enable early detection and appropriate treatment of particularly aggressive tumors at an early stage, when they are still potentially curable. There is the possibility of developing a “pick-up service” for samples, so that patients on “active surveillance” do not have to visit the clinic for sample acquisition. Instead, they would only need to appear in clinic for a follow-up examination when changes in the analyte marker profile are detected.

The method of this invention describes a non-invasive diagnosis and monitoring of prostate cancer. For this purpose, ejaculated prostate cells and immune cells are separated from mature sperm cells and analyzed in the case of semen samples. In the case of expressed prostatic secretions (EPS), prostate cells and immune cells are isolated from prostate massage urine samples and analyzed. Due to the increased mobility of metastatic cells, these appear in prostatic secretions of semen and expressed prostatic secretions (EPS). The use of established tumor markers allows tumor cells to be distinguished from benign cells. Since semen and expressed prostatic secretion (EPS) samples can be provided with a high frequency, this allows for close prostate cancer monitoring, so that disease progression to metastatic tumor stages can be detected in a timely manner. This can lead to a more favorable disease course (avoiding undertreatment), save money, and prevent unwanted side effects (avoiding overtreatment). Analyzing the immune cell composition in semen or ejaculate can provide information about the immune status of the tumor, enabling the development and monitoring of customized therapeutic approaches (personalized medicine).

Semen samples have not previously been considered an optimal analyte for making statements about processes in the prostate, as the sample is dominated by mature sperm cells and their precursor cells. However, ejaculation involves a regular, physiological activation and contraction of the prostate, producing a significant amount of prostate secretion, which accounts for a significant proportion of the semen sample (approximately 30%). Compared to analyzing disease-related changes in urine samples, this increased abundance of prostate secretions in semen may allow for a higher signal strength of analyzed disease markers and a lower variability of results when the sample is prepared accordingly. By separating mature sperm cells from the analyte, which is comprised of the low-density cell fraction with somatic cells from the semen, this method aims to concentrate disease-relevant markers and to eliminate meaningless signals (background). The claim of this invention thus is to provide the liquid biopsy with properties of a tissue biopsy so that it becomes a like a (quasi) non-invasive tissue biopsy.

The invention also relates to the use of an isolate from ejaculate, wherein the isolate is obtained by fractionating ejaculate into a low-density cell population and a high-density cell population, wherein the high-density cell population, which comprises mature sperm cells, is separated, and the isolate comprises the low-density cell population for diagnosing and/or monitoring prostate cancer and/or for monitoring the success of prostate cancer therapy.

The use of an isolate from expressed prostatic secretions (EPS) is also disclosed, wherein the isolate is obtained by fractionating expressed prostatic secretions (EPS) into a cellular fraction and a cell-poor (“paucicellular”) fraction, wherein the cell-poor fraction is separated off and the isolate comprises the cellular fraction, for diagnosing and/or monitoring prostate cancer and/or for monitoring prostate cancer treatment effectiveness.

The invention also relates to the use of an isolate of a cellular fraction and/or an isolate of a cell-poor (“paucicellular”) fraction, wherein the isolate is obtained by fractionating ejaculate from a vasectomized person into the cellular fraction and into the cell-poor fraction, for diagnosing and/or monitoring prostate cancer and/or for monitoring the success of prostate cancer therapy.

### DETAILED DESCRIPTION OF THE INVENTION

Embodiments within this document refer to all aspects of the invention and can be combined with each other as desired, unless the subject matter and description of the embodiments clearly indicate otherwise.

The term “one” or “a” is to be understood as “at least one” or “at least a,” unless the context clearly indicates otherwise.

The verbs “contain” and “comprise”, and their conjugations also include the verb “consist of” and its conjugations.

In a preferred embodiment, the method according to the invention is a method for diagnosing and/or monitoring prostate cancer.

In another preferred embodiment, the method according to the invention is a method for monitoring the success of a therapy for prostate cancer. A corresponding therapy does not include radical prostatectomy, as no ejaculate sample can be provided after this procedure. A corresponding therapy for prostate cancer can be selected, for example, from radiation, immunotherapy, androgen deprivation therapy, androgen receptor-targeted therapy (in particular with docetaxel and/or cabazitaxel), chemotherapy with a platinum compound (in particular with carboplatin), therapy with a systemic radiopharmaceutical (in particular with a therapeutic agent selected from radium-233, lutetium-177-PSMA-617), therapy with a PARP inhibitor (in particular with olaparib), or a combination of the aforementioned therapies. The radiation may be percutaneous radiation therapy, brachytherapy, or a combination thereof. In other words, the method may be a method for monitoring the success of a therapeutic procedure before deciding on radical prostatectomy. In other words, the method may be a method for monitoring the success of a therapeutic procedure other than radical prostatectomy.

In a preferred embodiment, the tumor-specific marker is a DNA, an RNA, or a protein, wherein the tumor-specific marker is preferably selected from prostate-specific membrane antigen (PSMA), prostate-specific antigen (PSA), prostate-specific acid phosphatase (PAP), prostein (SLC45A3, also P501S), homeobox protein Nkx-3.1 (NKX3.1), pyruvate kinase M2, tissue polypeptide antigen, thymidine kinase, phosphatase and tensin homolog (PTEN), androgen receptor (AR; including amplifications, mutations, and splice variants), estrogen receptors (ESR1, ESR2), keratins (KRT14, KRT15, KRT18, KRT19), kallikrein related peptidase 2 (KLK2), Schlafen 11 (SLFN11), breast cancer type 1 and 2 susceptibility proteins (BRCA1, BRCA2), plastin3 (PLS3), aldehyde dehydrogenase 1A1 (ALDH1A1), epithelial cell adhesion molecule (EPCAM), vimentin (VIM), cadherins (CDH1, CDH2), synaptophysin (SYP), chromogranin (CD56), and delta-like protein 3 (DLL3).

In a preferred embodiment, the analysis of the isolate comprises a method selected from transcriptome analysis, DNA sequencing, high-performance liquid chromatography (HPLC), mass spectrometry (MS), immunoassay methods, flow cytometry, and a method resulting from a combination of the above methods, wherein the analysis preferably comprises HPLC followed by MS.

According to the invention, an analysis is performed on an isolate, wherein a tumor-specific marker selected from a protein, DNA, and RNA is analyzed. Preferably, the tumor-specific marker selected from a protein, DNA, and RNA is quantified. In another embodiment, only a qualitative analysis is performed, i.e., it is analyzed whether a marker is found or not.

In a preferred embodiment, the analysis comprises at least one of the following steps selected from i) absolute quantification of the tumor-specific marker in the isolate and ii) relative quantification of the tumor-specific marker in the isolate relative to the total amount of protein or DNA or RNA in the isolate.

The isolate contains immune cells. In a preferred embodiment, the composition and differentiation of the immune cells are analyzed in the analysis of the isolate. Preferably, at least one marker selected from
markers for immune cell infiltration, preferably selected from CSF1, CXCL8, CXCR4, G-CSF, MCP-1, SDF-1,
T cell differentiation markers, preferably selected from CD3, CD4, CD8, CD25, CD28, CD40L, CTLA4, FasL, PD-1,
antigen presentation markers, preferably selected from MHC I, MHC II,
B cell differentiation markers, preferably selected from CD19, CD20, CD21, CD40, CD80, CD86,
granulocyte differentiation markers, preferably selected from CD11b, CD13, CD14, CD15, CD16, CD18, CD31, CD32, CD33, CD66b, CD117, CD123, CD125, CD170, CD193, Fcε receptors, myeloperoxidase,
macrophage differentiation markers, preferably selected from ARG1, CCR2, CD9, CD16, CD40, CD68, CD80, CD86, CD115, CD163, CD169, CD206, CD301, CSF1, CSF1R, CX3CR1, Dectin-1, F4/80, Fizz1, Lyve1, MARCO, NOS2, PDGF beta, PDL2, PPARG, TLR2, TLR4, TREM2,
inflammatory cytokines and chemokines, preferably selected from CCL2, CCL13, CCL18, IFN-γ, IL1a, IL1b, IL6, IL-1, IL-4, IL-5, IL-8, IL-10, IL-12, IL-13, TGF-β, TNF-α,

The result may allow further conclusions to be drawn about the malignancy and progression of a tumor as well as about a patient's immune defense.

In a preferred embodiment, the method comprises incubating the isolate with a specific antibody, wherein the specific antibody is optionally mass-labeled (mass spectrometry) or fluorophore-labeled (flow cytometry). In the case of mass spectrometric analysis, incubating the cells with a mass-labeled marker-specific (e.g., anti-PSMA or anti-PAP) antibody can raise the signal of the marker being sought to a level where there is no longer any background noise. This allows the signal to be “unmasked.” In the case of flow cytometry, a fluorophore-labeled antibody is recommended. It is possible that ejaculated prostate cancer cells in the semen sample are not present as individual cells and that they are detached from the tumor and expelled from the gland as a cell cluster instead. For the analysis of the cells via flow cytometry, an enzymatic digestion of the isolate can therefore be used for cell separation.

The method according to the invention may also comprise providing a sample of ejaculate or expressed prostatic secretions (EPS); for example, a patient sample may be provided by medical personnel or a clinic.

In a preferred embodiment, the method according to the invention comprises collecting a sample of ejaculate or prostate-massage (EPS) urine at a private location, preferably at the person's place of residence, wherein the fractionation is performed on the ejaculate or expressed urine of the person. As described, the non-invasive method with simple sample collection allows a “pick-up service” to be offered, meaning that patients do not have to visit a clinic in the first place, but only need to attend a follow-up examination if there is a change in the marker profile in their sample.

Herein, fractionation can be referred to as fractionating and vice versa.

Herein, centrifugation can be referred to as centrifuging and vice versa.

Further details concerning the first and second aspects in particular:

The first and second aspects of the method according to the invention relate to an isolate from ejaculate. The ejaculate is preferably human ejaculate. Preferably, the isolate from ejaculate originates from unfrozen material. In other words, the ejaculate was preferably not frozen prior to fractionation.

The isolate according to the first and second aspects is obtained or formed by fractionating ejaculate into a low-density cell population and a high-density cell population, wherein the high-density cell population, which comprises mature sperm cells, is separated and the isolate comprises the low-density cell population. The low-density cell population comprises somatic cells and gametic precursor cells of the sperm cells. Preferably, the fractionation comprises centrifugation. Centrifugation is performed, for example, at 200 g to 600 g, e.g., at about 400 g. When centrifuging the ejaculate, a cell pellet is preferably formed from the mature sperm cells and separated from the isolate. The remaining cell population contains the ejaculated somatic cells and gametic precursor cells of the sperm cells, in particular leukocytes, epithelial cells, fibroblasts, and other cells. The mature sperm cells have a higher density and can be separated from the isolate using the method from this invention; in particular, because they form a pellet during centrifugation.

Preferably, fractionation of ejaculate comprises centrifuging the ejaculate, whereby the low-density cell population is formed from somatic cells and low-density gametic precursor cells, wherein centrifuging the ejaculate results in enrichment of the somatic cells in the isolate and separation from the mature sperm cells. Enrichment may include an increase in the number of somatic cells per unit of volume of the isolate and/or an increase in the concentration of somatic cells relative to other cells.

Preferably, a centrifugation solution is used for centrifugation, wherein the centrifugation solution has a density that lies between the density of mature sperm cells and the density of somatic cells (leukocytes, epithelial cells, fibroblasts). This facilitates the separation of the low-density cell population (containing the somatic cell population) and the high-density cell population, which comprises mature sperm cells. Several centrifugation solutions may also be used, each having a different density, wherein at least one of the centrifugation solutions has a density that lies between the density of sperm cells and the density of somatic cells (the low-density cell population). If multiple such centrifugation solutions are used, centrifugation is performed using a density gradient, wherein the density gradient supports separation of the low-density cell population and the high-density cell population. In a preferred embodiment, the centrifugation solution contains ethylenediaminetetraacetate.

In a preferred embodiment, an inert dye is used during centrifugation of the ejaculate to visually distinguish between the low-density cell population and the high-density cell population. For example, when centrifuging the ejaculate, an inert dye may be used to visually distinguish between a phase containing the low-density cell population and a phase containing the high-density cell population.

In a preferred embodiment, after fractionation by centrifugation, the method comprises diluting the isolate and centrifuging to form a cell pellet before downstream analysis. The cell pellet contains somatic cells, in particular leukocytes, epithelial cells, and fibroblasts. When diluting the centrifugation solution, in particular, the density of the centrifugation solution is adjusted to the density of the low-density cell population, whereby a cell pellet can be formed particularly well during subsequent centrifugation. Centrifugation is carried out, for example, at 600 g to 2000 g, e.g., at approximately 1280 g. In a preferred embodiment, the cell pellet is fixed with paraformaldehyde for analysis.

The analysis is preferably performed on a cell pellet. In other words, the isolate is or comprises a cell pellet in preferred embodiments. A cell pellet has particular advantages because it contains the cells, DNA, RNA, and protein in high concentration and can be stored well or shipped for complex analyses.

In a preferred embodiment, a prostate massage is performed before the ejaculate is provided. In other words, in a preferred embodiment, the ejaculate comes from a person who has undergone a prostate massage before providing a sample of the ejaculate. This can increase the diagnostic value of the method.

In a preferred embodiment, the ejaculate is subjected to heat treatment at more than 25° C. prior to fractionation. The heat treatment is performed outside the human body. The heat treatment can be performed, for example, in a temperature range of more than 25° C. to 40° C., in particular at about 37° C. The heat treatment can be carried out, for example, over a period of 5 minutes to 60 minutes. The heat treatment promotes the physiologically intended liquefaction of the ejaculate. The heat treatment can improve sample preparation and analysis results.

Further details concerning in particular the third and fourth aspects:

The third and fourth aspects of the method relate to an isolate from expressed prostatic secretions (EPS).

Urine with expressed prostatic secretions (EPS) is also known as prostate massage urine. In other words, immediately before a urine sample was taken, a prostate massage was performed on the person from whom the urine with expressed prostatic secretions (EPS) originated. Preferably, the person is a human being. Preferably, it is human prostate massage urine.

The isolate from expressed prostatic secretions (EPS) is obtained by fractionating the prostate massage urine into a cellular fraction and a cell-poor fraction, whereby the cell-poor fraction is separated off and an isolate from expressed prostatic secretions (EPS) is formed, wherein the isolate comprises the cellular fraction. The cells of the cellular fraction originate essentially from the prostate gland and urinary tract. The cell-poor (“paucicellular”) fraction essentially comprises the soluble fraction of the urine and prostate secretion. The cell-poor fraction is preferably a cell-free fraction.

Preferably, the isolate from prostate massage urine originates from unfrozen material. In other words, preferably the prostate massage urine was not frozen prior to fractionation.

Fractionation preferably comprises centrifugation. Preferably, the isolate forms a cell pellet during centrifugation. Centrifugation is carried out, for example, at 600 g to 2000 g, e.g., at about 1280 g. In a preferred embodiment, the cell pellet is fixed with paraformaldehyde for analysis.

The analysis is preferably performed on a cell pellet. In other words, the isolate is or comprises a cell pellet in preferred embodiments. A cell pellet has particular advantages because it contains the cells, DNA, RNA, and protein in high concentration and can be stored well or shipped for complex analyses.

Further embodiments relating in particular to the fifth and sixth aspects:

The fifth and sixth aspects of the method according to the invention relate to an isolate from the ejaculate of a vasectomized person, wherein the isolate was obtained by fractionating the ejaculate of a vasectomized person into a cellular fraction and a cell-poor fraction. The person is preferably a human being. The ejaculate is preferably human ejaculate.

In a vasectomy, the connection between the testicle and the ejaculatory duct is severed so that no sperm cells or gametic precursor cells can enter the semen during ejaculation. In the embodiments according to the fifth and sixth aspects (after vasectomy), the process steps for separating mature sperm cells from the ejaculate are not required. In addition, the ejaculate also does not contain any gametic precursor cells, which can further improve the diagnostic value of the method.

Preferably, the isolate from ejaculate comes from unfrozen material. In other words, preferably the ejaculate was not frozen prior to fractionation.

Fractionation preferably involves centrifugation. Preferably, the isolate of the cellular fraction forms a cell pellet during centrifugation. Centrifugation is carried out, for example, at 600 g to 2000 g, e.g., at approximately 1280 g. In a preferred embodiment, the cell pellet is fixed with paraformaldehyde for analysis.

The analysis of the isolate of the cellular fraction is preferably performed on a cell pellet. In other words, the isolate of the cellular fraction is or comprises a cell pellet in preferred embodiments. A cell pellet has particular advantages because it contains the cells, DNA, RNA, and protein in high concentration and can be stored well or shipped for complex analyses.

In a preferred embodiment of the method according to the invention, a vasectomy is performed before the ejaculate is provided.

In a preferred embodiment, a prostate massage is performed before the ejaculate is provided. In other words, in a preferred embodiment, the ejaculate comes from a person who has undergone a prostate massage before providing a sample of the ejaculate. This can increase the diagnostic value of the method.

In a preferred embodiment, the ejaculate is subjected to heat treatment at more than 25° C. prior to fractionation. The heat treatment is performed outside the human body. The heat treatment can be performed, for example, in a temperature range of more than 25° C. to 40° C., in particular at about 37° C. The heat treatment can be carried out, for example, over a period of 5 minutes to 60 minutes. The heat treatment promotes the physiologically intended liquefaction of the ejaculate. The heat treatment can improve sample preparation and analysis results.

The invention is illustrated below by means of examples, which are not intended to be limiting.

### Example 1

### a) Sample Collection

Sample collection: Patients with prostate carcinoma (PCa) provide a sample of ejaculate (semen) at home or, alternatively, during a scheduled routine examination at the urology outpatient clinic of the treating hospital.

Optional—prostate massage before sample collection: In order to enable increased mobilization of epithelial cells from the glandular ducts of the prostate into the ejaculate, the physician may perform a prostate massage before ejaculate collection. This can be done as part of the regular scheduled digital rectal exam (DRE) of the prostate.

b) Isolation of Somatic Cells from the Ejaculate Sample

Next, the low-density somatic and gametic cells are extracted from the ejaculate sample. Since liquid biopsies degrade before fixation, the step described is time-sensitive and is preferably performed at the site of sample collection. The steps after sample fixation are less time-sensitive, so the sample can then be sent to the central laboratory.

Physiological liquefaction of the ejaculate sample: When extracting the cells, the ejaculate sample is first incubated in a heating cabinet (37° C.) for 15 minutes, which causes the liquid biopsy to liquefy physiologically. This frees the ejaculated cells from the protein matrix of the ejaculate, which facilitates the separation of the sample into different cell fractions during the next step.

Separation of the sample by centrifugation in a density gradient: The liquefied ejaculate sample is loaded onto a density gradient made of an inert material with no physiological influence on cell physiology. A density gradient is classically produced by layering several solutions with different densities, starting with the highest density and using a lower density with each layer (e.g., 90%, 70%, 50%, 30% concentration). When layering the gradient, turbulence and mixing of the resulting phases should be avoided as much as possible. The subsequent centrifugation takes place at a very low speed (˜400 g) so that the gradient is maintained. The cells follow the centrifugal force towards the bottom of the vessel, where they remain in the phase corresponding to their own density due to the low forces.

Optional—selection of a uniform density for the gradient solution: Since this procedure is primarily concerned with the separation of sperm cells and the test should ideally be transportable and not prepared on site, a solution with a uniform density is recommended. In this case, the selected density of the solution lies between the density of sperm cells and the other cells in the sample. When the ejaculate sample is centrifuged in this solution, the sperm cells form a pellet (higher density than the solution) and the remaining cells accumulate in the phase between the seminal plasma and the gradient solution (lower density than the solution). Strictly speaking, after this modification, it is no longer a density gradient in the classical sense.

Cooling during the process steps before fixation: Since the cells are removed from their biological environment during processing, changes from their original state in the prostate are to be expected. Cooling the sample slows down these undesirable processes. For this reason, a cooled solution (4° C.) can be used once the sample has been removed from the incubator (37° C.). The reaction vessels of the assay, including the density gradient solution, can therefore be stored in the refrigerator (4° C.) before the test is performed. A refrigerated centrifuge would further improve the result, but it cannot be assumed that all urology clinics have a refrigerated centrifuge.

Optional—Use of chelating agents or protease inhibitors in density gradient solution: Chelating agents (e.g., ethylenediaminetetraacetate; EDTA) and protease inhibitors in the solution can further limit the enzymatic activity in the sample and thus slow down the degradation of the sample. However, their effect can have adverse effects on subsequent steps in the procedure (e.g., reduced antibody binding capacity or cell separation). For this reason, the use of these reagents may be avoided under certain circumstances.

Procedure protocol: The sample preparation described below can be carried out using a test kit, which is designed to make the procedure as intuitive and error-free as possible. The solutions in the kit that are used before the fixation step can be stored in the refrigerator (4° C.) before use:
1) Layer the ejaculate sample (˜2-6 mL) on 9 mL of Histopaque solution (90% Histopaque®1077, 10% phosphate-buffered saline; PBS) in a 15 mL Falcon tube;
2) Centrifuge the Falcon tube for 30 min at 400 g (room temperature=RT);
3) Remove 9 mL of the supernatant from the solution-air phase from top to bottom in circular movements; the resulting pellet must not be swirled to avoid transferring sperm cells into the isolate;
4) Transfer the 9 mL supernatant to a 50 mL Falcon tube containing 41 mL PBS;
5) Mix the cell suspension by inverting the Falcon tube several times (final concentration: 16% Histopaque®1077, 84% PBS).
6) Centrifuge the Falcon tube for 30 min at 1,280 g (RT).
7) Discard the supernatant, ensuring that the pellet does not fall dry.
8) Resuspend the cell pellet in 0.5 mL PFA-PBS (4% paraformaldehyde; PFA; RT);
9) Incubate the cell suspension for 15 min for PFA fixation (RT);
10) Stop fixation by adding 40 mL PBS (4° C.) and inverting the Falcon tube several times;
11) Centrifuge the sample in the Falcon tube for 30 min at 1,280 g (RT).
12) Remove the diluted PFA-PBS solution, resuspend the cell pellet in 1 mL PBS (4° C.);
13) Send the sample (=the isolate) to the central laboratory (4° C.).

### c) Analysis of the Isolate

In the central laboratory, the fixed samples are examined for prostate cancer (PCa) specific tumor markers (PCa markers).

### Variant 1: Flow Cytometry

Double labeling of the isolated cells against the PCa markers PSMA and PAP: It is possible to label formaldehyde-fixed cells with antibodies for flow cytometry. The fixed sample is stored and processed in a cool environment (4° C.) at the central laboratory. In this example, the cells are double-labeled with two fluorescence-labeled PCa markers (in this example: prostate-specific membrane antigen, PSMA; prostate-specific acid phosphatase, PAP). However, this example is not restrictive and other PCa markers may be used. Sample preparation is carried out according to established protocols.

Flow cytometry results: When collecting data via flow cytometry, the fluorescence-labeled cells are passed through a capillary with a very small diameter. The sensor registers a change in light refraction when a cell passes it. Flow cytometry allows to measure the size (forward scatter; FSC) and granularity (side scatter; SSC) of the cells under investigation. These values alone can already be of prognostic value for the patient. In addition, the PCa markers mentioned above can be detected with fluorescence-labeled antibodies, because this changes the refractory pattern of cells passing the sensor. When evaluating FSC, SSC and fluorescence in the resulting dataset, distinct cell populations can be identified, analyzed and compared between patients and disease states. The cell subset testing positive for the analyzed antigens can be calculated as percentage of the total analyzed cell population. Example result:
1) 2% of the cells in the sample are PAP-positive (PAP+);
2) 5% of the cells in the sample are PSMA-positive (PSMA+);
3) 1.5% of the cells in the sample are both PAP- and PSMA-positive (PAP+ PSMA+).

By recording the percentage of PAP+ and PSMA+ cells in the sample, a snapshot is obtained of those mobile prostate cancer cells that have been released from the local tumor and released into the patient's ejaculate. These represent highly mobile tumor cells that can infiltrate not only the ejaculate, but also the patient's neighboring lymph nodes and organs. For this reason, the present method allows to evaluate the patient's risk of metastasis.

### Variant 2: Proteome Analysis of the Isolated Cells

HPLC-MS on fixed pellets of isolated somatic cells: The isolated, fixed cell pellet is prepared according to established protocols and analyzed using high-performance liquid chromatography (HPLC) followed by mass spectrometry (MS). HPLC allows separation into different protein fractions, which are analyzed using downstream MS. The method is established, commercially available, and, provided the starting material is of good quality, allows a protein spectrum with a resolution of over 4000 different proteins.

Optional—labeling of the sample with gold-associated antibodies: The proteome of a sample consists of highly abundant proteins and less abundant proteins, which can cause problems in proteome analysis. The signals of the PCa markers can be masked by the background noise of the highly abundant proteins if they have a similar mass. This problem can be countered by prior incubation of the fixed cell pellets with gold-labeled antibodies that bind the PCa markers PSMA and PAP. Due to the high mass of the gold particles, the signals of the antibodies are raised to a mass range in MS where there is only very low background noise. This significantly increases the sensitivity of the method, allowing PCa markers to be quantified even at low abundance.

Quantification of the datasets obtained: The method described in this example initially focuses on the quantification of two established PCa markers (prostate-specific membrane antigen, PSMA; prostate-specific acid phosphatase, PAP). However, this example is not limiting and other PCa markers can be used. There are several established ways to quantify the signal strength of the marker proteins in the sample.

d) Comparison of Results with Established Reference Values

In order to evaluate the results obtained from an individual examination, they can be compared with a reference. For this purpose, the data set from a previously conducted clinical study is used, in which a statistically significant number of patients were examined with the method.

Case-control study to establish reference values: For a clinical study, various clinical parameters (i.e., PSA value, Gleason score, TNM classification after prostatectomy, age) are collected and correlated with the measured results of the method according to the invention. The study population and exemplary results of the study are illustrated below, whereby, for better comprehensibility, the results of flow cytometry are compared with the Gleason score from an core needle biopsy of the patient in the example presented. Another example could be described using the results of the alternatively described proteome analysis (instead of flow cytometry) and using other clinical parameters (e.g., PSA value, age, decision to undergo prostatectomy, etc.).

Patient collective (Gleason score) of the case-control study:

Cohort 1: 100 “low risk” patients with a low risk of metastasis (well-differentiated biopsy tissue, Gleason score 6).

Cohort 2: 100 “high risk” patients with a high risk of metastasis (poorly differentiated biopsy tissue, Gleason score 8-9).

The test results on the patient cohort lead to the establishment of reference values (percentage of PAP+ and PSM+ cells; determined by flow cytometry of isolated somatic cells from ejaculate), e.g.:

| | | | PAP+ |
|---|---|---|---|
| | PAP+ | PSMA+ | PSMA+ |
| “Low risk” patients (n = 100) | 1%-5% | 2%-6% | 0%-1% |
| High-risk patients (n = 100) | 5%-10% | 6%-12% | 1%-5% |

Comparison of individual test results with established reference values (hypothetical case); In February 2024, a core needle biopsy was taken from a patient with prostate cancer, and the examination revealed that his prostate cancer was indolent and relatively well differentiated (Gleason 6). Prior to the biopsy, the patient provided an ejaculate sample, which was analyzed using the method described in the invention. A comparison of the results with established reference values shows that the patient's sample is within the range typical for his tumor stage (specifically: 2% PAP+, 3% PSMA+, 1% PAP+ PSMA+). Two months later, the patient provides another ejaculate sample during a follow-up examination. The sample is still within the defined reference range and does not differ significantly in composition from the previous sample. The patient's ejaculate values continue to be monitored every three months, and there are no indications of progression of his prostate cancer over a longer period of time. After three years however, the composition of the cells in the ejaculate changes (specifically: 8% PAP+, 12% PSMA+, 5% PAP+ PSMA+), although the PSA level in the blood has not changed. For this reason, another biopsy is ordered. The biopsy results show that the tumor has now progressed to a more aggressive stage (Gleason 8). During prostatectomy, the lymph nodes are removed and examined pathologically. No tumor infiltrates are found in the lymph nodes because the progression was noticed promptly due to the increased tumor cell representation in the ejaculate.

### e) Application of the Procedure in Clinical Practice

Application of the method in active surveillance: By comparing the values obtained with the reference values determined in clinical studies, a meaningful risk score can be determined that indicates the probability of the tumor progressing to a more aggressive stage. As this is a cost-effective, non-invasive diagnostic procedure, longitudinal observation of tumor development is possible and planned. The following table illustrates the possible course of such longitudinal observation of the disease progression:

| | January | March | May | July | September | November | January | March |
|---|---|---|---|---|---|---|---|---|
| | 2025 | 2025 | 2025 | 2025 | 2025 | 2025 | 2026 | 2026 |
| PAP+ (%) | 2 | 3 | 6 | 2 | 6 | 5 | 12 | 14 |
| PSMA+ (%) | 5 | 4 | 3 | 5 | 8 | 7 | 16 | 13 |
| PSA+ | 1 | 1 | 1 | 2 | 4 | 3 | 5 | 6 |
| PSMA+ | | | | | | | | |
| (%) | | | | | | | | |
| Clinical | Wait | Wait | Wait | Wait | Wait | Wait | Wait | Prostatectomy |
| decision | | | | | | | | |

In addition to this test, the results of several other diagnostic methods (e.g., blood PSA level, DRE, general condition, biopsy findings) can be integrated into the doctor's decision-making process in clinical practice. After surgical removal of the prostate, this procedure is no longer applicable, as no ejaculate sample can be obtained. This limits the available patient population for the method to patients before radical prostatectomy.

### Application of the Procedure for Monitoring the Success of Therapeutic Approaches Excluding Radical Prostatectomy:

The procedure is not only suitable for monitoring PCa in the active surveillance approach, but also for monitoring the success of therapeutic approaches excluding radical prostatectomy (e.g., radiation or immunotherapy). In this case, a reduced or increased release of PCa cells into the ejaculate is possible after therapeutic intervention (e.g., due to increased cell death or increased phagocytosis). An example of the development of the detected cell counts is illustrated here:

| | January | March | May | July | September | November | January | March |
|---|---|---|---|---|---|---|---|---|
| | 2026 | 2026 | 2026 | 2026 | 2026 | 2026 | 2027 | 2027 |
| PAP+ (%) | 12 | 14 | 12 | 30 | 6 | 5 | 2 | 3 |
| PSMA+ (%) | 16 | 13 | 16 | 55 | 8 | 7 | 5 | 4 |
| PSA+ | 5 | 6 | 5 | 10 | 4 | 3 | 1 | 1 |
| PSMA+ | | | | | | | | |
| (%) | | | | | | | | |
| Clinical | Wait | Wait | Radiation | Wait | Wait | Waiting | Waiting | Waiting |
| decision | | | | | | | | |

As part of this monitoring of the therapeutic interventions, it may also be beneficial to observe inflammation markers, especially since established therapeutic approaches influence the immune status within the tumor.

### Example 2

The experimental procedure in this example corresponds to the experimental procedure in Example 1, except that the isolate does not come from ejaculate but from prostate massage urine. The preceding sample preparation before the analysis step is simpler because prostate massage urine does not contain sperm cells.

### a) Sample Collection

Sample collection: PCa patients provide a first-void urine sample at home or during a scheduled routine visit at the urologist.

### b) Isolation of Prostate Secretions

The prostate secretion is then obtained. Since liquid biopsies degrade before fixation, the step described is time-sensitive and is preferably carried out at the place where the sample is taken. The steps after sample fixation are less time-sensitive, so that the sample can then be sent to the central laboratory.

Cooling of the procedure steps prior to fixation: Since the cells are removed from their biological environment during processing, changes from their original state in the prostate are to be expected. Cooling the sample slows down these undesirable processes.

Procedure protocol: The sample preparation described below can be carried out using a test kit provided, which is designed to make the procedure as intuitive and error-free as possible. The solutions in the kit that are used before the fixation step can be stored in the refrigerator (4° C.) before use:
1) Centrifuge the expressed urine sample in a 50 mL Falcon tube for 5 min at 1,280 g (4° C.). Due to its higher protein content, the expressed prostatic secretions (EPS) in prostate massage urine has a higher density than the urine fraction of the sample. Centrifugation separates the EPS from the urine in the sample.
2) Discard the supernatant (urine), leaving the EPS in the Falcon tube.
3) Dilute the EPS with 10 mL PBS (4° C.) by pipetting up and down;
4) Centrifuge the EPS sample in a Falcon tube for 30 min at 1,280 g (RT);
5) Discard the supernatant (soluble EPS fraction), ensuring that the cellular fraction (pellet) does not fall dry;
6) Resuspend the cell pellet in 1 mL PFA-PBS (4% paraformaldehyde; PFA; RT);
7) Incubate the cell suspension for 15 min for PFA fixation (room temperature; RT).
8) Stop fixation by adding 40 mL PBS (4° C.) and inverting the Falcon tube several times;
9) Centrifuge the sample in the Falcon tube for 30 min at 1,280 g (4° C.);
10) Remove the diluted PFA-PBS solution, resuspend the cell pellet in 1 mL PBS (4° C.);
11) Send the sample (=the isolate) to the central laboratory (4° C.).

### c) Analysis of the Isolate

The analysis is performed as in Example 1, for example.

### Example 3

The test procedure in this example corresponds to the test procedure in Example 1, except that the isolate does not originate from physiologically unaltered ejaculate (hereinafter referred to as “native”), but from ejaculate after vasectomy of the patient (hereinafter referred to as “vasectomy ejaculate”). The sample preparation before analysis is simpler, as this ejaculate does not contain any sperm cells or gametic precursor cells.

### a) Vasectomy

A vasectomy is a borderline case of medical intervention. It is usually a lifestyle decision and is not covered by statutory health insurance in Germany. An exception is made if there is a proven medical reason for the vasectomy. For example, health insurance companies will cover the cost of a vasectomy if the patient's partner is not allowed to become pregnant for medical reasons and other methods of contraception are not feasible or reasonable. In the present case, there is a valid medical reason for the vasectomy. Men with prostate cancer generally no longer wish to have children, which means that this aspect should not pose an obstacle to the procedure. The vasectomy can be performed as part of a prostate biopsy to investigate suspected prostate cancer.

### b) Sample Collection

Sample collection: Vasectomized PCa patients submit an ejaculate sample at home or, alternatively, during a scheduled routine examination at the urology outpatient clinic of the treating hospital.

c) Isolation of Ejaculated Cells and Prostate Secretions from Vasectomy Ejaculate

In the next step, the ejaculated cells and prostate secretions are isolated from the sample. The prostate secretions differ from the seminal plasma of a native ejaculate sample, as there has been no contribution from the testes. It is comparable to expressed prostatic secretions (EPS) after prostate massage. However, in this case, a significantly higher amount of prostate secretion and cells is released than in expressed urine, and there is no contribution from the urinary bladder. For this reason, from a diagnostic point of view, this is a higher-quality liquid biopsy with increased abundance, which allows for greater sensitivity of downstream methods than when working with prostate massage urine.

Since liquid biopsies degrade before fixation, the step described is time-sensitive and is preferably performed at the site of sample collection. The steps after sample fixation are less time-sensitive, so that the sample can then be sent to the central laboratory.

Cooling of the procedural steps prior to fixation: Since the cells are removed from their biological environment during processing, changes from their original state in the prostate are to be expected. Cooling the sample slows down these undesirable processes.

Procedure protocol: The sample preparation described below can be carried out using a test kit provided, which is designed to make the procedure as intuitive and error-free as possible. The solutions in the kit that are used before the fixation step can be stored in the refrigerator (4° C.) before use:
1) Centrifuge the vasectomy ejaculate sample in a 15 mL Falcon tube for 30 min at 1,280 g (RT).
2) Transfer the supernatant (prostate secretion) to a new 15 mL Falcon tube, leaving the ejaculated cells as a pellet in the tube.
3) Proceed with steps 4-9 using the isolated cells. Freeze the isolated soluble phase of the prostate secretion (−20° C.).
4) Resuspend the cell pellet in 1 mL PFA-PBS (4% paraformaldehyde; PFA; RT).
5) Incubate the cell suspension for 15 min for PFA fixation (room temperature; RT);
6) Stop fixation by adding 14 mL PBS (4° C.) and inverting the Falcon tube several times;
7) Centrifuge the sample in the Falcon tube for 30 min at 1,280 g (RT);
8) Remove the diluted PFA-PBS solution and resuspend the cell pellet in 1 mL PBS (4° C.).
9) Send the samples (=the isolates) with the isolated cells (4° C.) and the prostate secretion (−20° C.) to the central laboratory.

### c) Analysis of the Isolates

The isolated cells are analyzed as in Example 1, for example. The isolated prostate secretion is analyzed as in Example 1, whereby the option of flow cytometry is not feasible per definition (cell-free soluble fraction). Instead, HPLC-MS is preferred.

What is claimed is:

1. A method for analyzing a tumor-specific marker, wherein the method comprises:
performing an analysis on an isolate, wherein the tumor-specific marker selected from a protein, DNA, and RNA is analyzed, wherein the isolate was obtained by fractionating an ejaculate into a low-density cell population and a high-density cell population, wherein the high-density cell population comprising mature sperm cells was separated and the isolate comprises the low-density cell population, wherein the isolate comprises a cell pellet.

2. The method of claim 1, wherein the method is a method for diagnosing and/or monitoring prostate cancer.

3. The method of claim 1, wherein the method is a method for monitoring a success of a therapy of prostate cancer.

4. The method of claim 1, wherein the method comprises fractionating of ejaculate into a low-density cell population and a high-density cell population, wherein the high-density cell population, which comprises the mature sperm cells, is separated and the isolate comprises the low-density cell population.

5. The method of claim 1, wherein the fractionating of ejaculate comprises centrifuging of the ejaculate.

6. The method of claim 1, wherein the ejaculate is subjected to heat treatment at more than 25° C. prior to the fractionating.

7. The method of claim 5, wherein a low-density cell population is formed comprising somatic cells and low-density gametic precursor cells, wherein the centrifuging of the ejaculate results in enrichment of the somatic cells in the isolate and separation from the mature sperm cells, wherein a centrifugation solution is used for the centrifuging, and wherein the centrifugation solution has a density between a density of the mature sperm cells and densities of the somatic cells and the gametic precursor cells.

8. The method of claim 5, wherein an inert dye is used during the centrifuging of the ejaculate for optical differentiation between the low-density cell population and the high-density cell population.

9. The method of claim 5, wherein a cell pellet is formed from the mature sperm cells and separated from somatic cells and gametic precursor cells during the centrifuging of the ejaculate.

10. The method of claim 7, wherein, after the fractionating by centrifuging using the centrifugation solution and before performing the analysis, the method comprises: diluting the centrifugation solution and centrifuging, such that a cell pellet is formed.

11. The method of claim 1, wherein the ejaculate is from a person who underwent prostate massage before providing a sample of the ejaculate.

12. The method of claim 1, wherein a sample of the ejaculate is collected at a private location, wherein the fractionating is performed on the ejaculate of the person.

13. The method of claim 1, wherein the tumor-specific marker is chosen from prostate-specific membrane antigen (PSMA), prostate-specific antigen (PSA), prostate-specific acid phosphatase (PAP), prostein (SLC45A3, also P501S), homeobox protein Nkx-3.1 (NKX3.1), pyruvate kinase M2, tissue polypeptide antigen, thymidine kinase, phosphatase and tensin homolog (PTEN), androgen receptor (AR; including amplifications, mutations, and splice variants), estrogen receptors (ESR1, ESR2), keratins (KRT14, KRT15, KRT18, KRT19), kallikrein related peptidase 2 (KLK2), Schlafen 11 (SLFN11), breast cancer type 1 and 2 susceptibility proteins (BRCA1, BRCA2), plastin3 (PLS3), aldehyde dehydrogenase 1A1 (ALDH1A1), epithelial cell adhesion molecule (EPCAM), vimentin (VIM), cadherins (CDH1, CDH2), synaptophysin (SYP), chromogranin (CD56), and delta-like protein 3 (DLL3).

14. The method of claim 1, wherein at least one of the following markers is analyzed during analysis of the isolate:
markers for immune cell infiltration (CSF1, CXCL8, CXCR4, G-CSF, MCP-1, SDF-1),
T-cell differentiation markers (CD3, CD4, CD8, CD25, CD28, CD40L, CTLA4, FasL, PD-1),
antigen presentation markers (MHC I, MHC II),
B-cell differentiation markers (CD19, CD20, CD21, CD40, CD80, CD86),
granulocyte differentiation markers (CD11b, CD13, CD14, CD15, CD16, CD18, CD31, CD32, CD33, CD66b, CD117, CD123, CD125, CD170, CD193, Fcε receptors, myeloperoxidase),
macrophage differentiation markers (ARG1, CCR2, CD9, CD16, CD40, CD68, CD80, CD86, CD115, CD163, CD169, CD206, CD301, CSF1, CSF1R, CX3CR1, Dectin-1, F4/80, Fizz1, Lyve1, MARCO, NOS2, PDGF beta, PDL2, PPARG, TLR2, TLR4, TREM2),
inflammatory cytokines and chemokines (CCL2, CCL13, CCL18, IFN-γ, IL1a, IL1b, IL6, IL-1, IL-4, IL-5, IL-8, IL-10, IL-12, IL-13, TGF-β, TNF-α).

15. A use of an isolate from ejaculate, wherein the isolate comprises a cell pellet, wherein the isolate is obtained by fractionating ejaculate into a low-density cell population and a high-density cell population, wherein the high-density cell population, which comprises mature sperm cells, is separated and the isolate comprises the low-density cell population, for diagnosing and/or monitoring prostate cancer and/or for monitoring a success of therapy of prostate cancer.
